(19) United States
(12) Patent Application Publication
(10) Pub. No.: US 2020/0008666 A1
(43) Pub. Date: Jan. 9, 2020

(54) METHODS AND SYSTEMS FOR VISION MONITORING

(71) Applicant: Tarseer, Inc., Bronx, NY (US)

(72) Inventors: Jorge ANTON GARCIA, Madrid (ES); Andre C. NEWLAND, Bronx, NY (US); Erika A. MARMOL, Dublin, CA (US); Terence TUFUOR LARTEY, Brighton, MA (US); Alfonso ANTON LOPEZ, Barcelona (ES); Julian Augustus FROST, Bronx, NY (US); Michael Paul TATUM, JR., Chester, VA (US)

(21) Appl. No.: 16/458,602

(22) Filed: Jul. 1, 2019

Related U.S. Application Data
(60) Provisional application No. 62/693,711, filed on Jul. 3, 2018.

(51) Int. Cl. A61B 3/00; A61B 3/113; A61B 5/11; A61B 5/0484; A61B 3/12; A61B 3/18; A61B 3/024

(52) U.S. Cl. CPC A61B 3/0091; A61B 3/0008; A61B 3/005; A61B 3/0058; A61B 3/113; A61B 5/0004; A61B 3/0033; A61B 5/04842; A61B 3/12; A61B 3/18; A61B 3/024; A61B 5/1114

(57) ABSTRACT

An apparatus for measuring a visual field of a subject can include an enclosure, wherein the enclosure surrounds at least one eye of the subject; a display comprising a distance from the eye of the subject to a surface of the display, wherein the distance is substantially constant along a portion of the surface of the display; and a plurality of light sources disposed on the display, wherein the light sources comprise a plurality of stimuli for measuring the visual field of the subject, wherein the apparatus may be worn by the subject.

### CROSS-REFERENCE

This application claims the benefit of U.S. Provisional Patent Application No. 62/693,711 filed Jul. 3, 2018, entitled “Methods and Systems for Vision Monitoring” [Atty. Ref. No. 53496-701.101], which is incorporated by reference herein in its entirety.

### BACKGROUND

Current apparatuses and methods for testing the visual field of a subject may be less than ideal in at least some respects, among which may be unreliability and/or inaccessibility. For example, the testing apparatuses may be expensive, highly dependent on patient performance, and/or non-portable. Additionally, to obtain accurate results, the testing methods may require personalized supervision from a trained technician in a controlled environment. In another example, test-takers may have difficulty concentrating during a testing procedure, which may last up to several minutes. Test-takers may also become nervous or frustrated during the testing procedure, which may skew the test results.

### SUMMARY

Accordingly, there exists unmet need for improved apparatuses and methods which address at least some of the abovementioned problems. Improved apparatuses and methods may, for example, involve less subject attention and/or time, not involve a technician, be used remotely, provide more accurate and precise results, and/or be more affordable.

In an aspect, an apparatus for measuring visual function across a visual field of a subject is disclosed. In some embodiments, the apparatus may comprise: an enclosure, wherein the enclosure is configured to surround at least one eye of the subject; a display comprising a surface, wherein the surface is configured to be spared a distance from the eye of the subject, wherein the distance is substantially constant along a portion of the surface of the display; and a plurality of light sources disposed on the display, wherein the light sources comprise a plurality of stimuli for measuring the visual field of the subject, wherein the apparatus is configured to be worn by the subject.

In some embodiments, the surface of the display comprises a dome shape. In some embodiments, the surface of the display comprises a shape which is hemispherical. In some embodiments, the surface of the display comprises a shape which is a portion of the surface of a sphere. In some embodiments, the apparatus is monocular. In some embodiments, the apparatus is binocular. In some embodiments, the apparatus is light sealed. In some embodiments, the apparatus is semitransparent. In some embodiments, the display comprises a plurality of displays.

In some embodiments, the plurality of light sources comprises light emitting diodes. In some embodiments, the display does not comprise a back-light. In some embodiments, the plurality of light sources is stationary relative to the surface of the display. In some embodiments, the plurality of light sources is mobile relative to the surface of the display.

In some embodiments, the plurality of light sources additionally comprises a plurality of fixation points. In some embodiments, illumination of a first light source comprises a first fixation point, wherein illumination of a second light source comprises a stimulus, and wherein the second light source comprises a second fixation point after the first light source is extinguished. In some embodiments, a first light source is disposed apart from a second light source on the surface of the display. In some embodiments, the plurality of light sources comprises locations on a map of the visual field.

In some embodiments, the display comprises an eye tracker configured to measure a point of gaze of the subject. In some embodiments, the eye tracker comprises an optical tracking system. In some embodiments, the optical tracking system is adapted to record a corneal reflection. In some embodiments, the optical tracking system is adapted to record a pupil position. In some embodiments, the display comprises a head-motion tracking system. In some embodiments, the display comprises one or a plurality of light sources configured to control ambient light level.

In some embodiments, a response to the stimulus is characterized by a change from a first point of gaze to a second point of gaze. In some embodiments, a response to the stimulus is characterized by a press of a button. In some embodiments, a response to the stimulus is characterized by electroencephalography.

In some embodiments, the apparatus is configured to characterize a state or condition of the eye of the subject. In some embodiments, the state or condition of the eye of the subject comprises glaucoma. In some embodiments, the state or condition of the eye of the subject comprises stroke. In some embodiments, the state or condition of a subject eye comprises brain tumor. In some embodiments, the state or condition of a subject eye comprises a rate of vision loss. In some embodiments, the state or condition of each eye is characterized separately. In some embodiments, the state or condition of both eyes is characterized simultaneously.

In some embodiments, the apparatus further comprises an ophthalmoscope. In some embodiments, the apparatus is portable. In some embodiments, the apparatus comprises one or a plurality of lens, the one or a plurality of lenses is configured to focus light onto a retina of the subject. In some embodiments, the apparatus does not include a lens. In some embodiments, a set of visual field data is configured to be transmitted to a mobile device. In some embodiments, the mobile device is configured to provide a map of the visual field. In some embodiments, the map of the visual field is configured to be provided to the subject or a third party.

In another aspect, a method of measuring visual function across a visual field of a subject is disclosed. In some embodiments, the method comprises: providing a head-mounted display comprising a plurality of light sources thereon, wherein the head-mounted display is configured to enclose an eye of the subject; wherein the head-mounted display is configured to be spaced a distance from the eye of the subject, the distance comprising a radius of an arc along a surface of the display; illuminating a first light source, wherein the first light source comprises a first fixation point; illuminating a second light source, wherein the second light source comprises a first stimulus; recording a response to a first stimulus; and illuminating a third light source.

In some embodiments, the third light source comprises the second light source and the second light source comprises a second fixation point after the first light source is extinguished. In some embodiments, the method further comprises extinguishing the first light source. In some embodiments, the method further comprises extinguishing the second light source. In some embodiments, the third light source is displaced away from the first light source and the second light source and wherein the third light source comprises a second fixation point after the first light source is extinguished.

In another aspect, an apparatus for measuring visual function across a visual field of a subject is disclosed. In some embodiments, an apparatus comprises: a head-mounted display comprising: an enclosure, wherein the enclosure is configured to surround at least one eye of the subject; a plurality of light sources disposed on the head-mounted display, wherein the head-mounted display is configured to be spaced a distance from the eye of the subject, the distance comprising a radius of an arc along a surface of the head-mounted display; and a plurality of light sources disposed on the head-mounted display, wherein the light sources comprise a plurality of stimuli for measuring the visual field of the subject.

In some embodiments, the surface of the head-mounted display comprises a dome shape. In some embodiments, the surface of the head-mounted display comprises a shape which is hemispherical. In some embodiments, the surface of the head-mounted display comprises a shape which is a portion of the surface of a sphere. In some embodiments, the apparatus is monocular. In some embodiments, the apparatus is binocular. In some embodiments, the apparatus is light sealed. In some embodiments, the apparatus is semitransparent.

In some embodiments, the plurality of light sources comprises light emitting diodes. In some embodiments, the head-mounted display does not comprise a back-light. In some embodiments, the plurality of light sources is stationary relative to the surface of the head-mounted display. In some embodiments, the plurality of light sources is mobile relative to the surface of the head-mounted display.

In some embodiments, the plurality of light sources additionally comprises a plurality of fixation points. In some embodiments, illumination of a first light source comprises a first fixation point, and illumination of a second light source comprises a stimulus, and wherein the second light source comprises a second fixation point after the first light source is extinguished. In some embodiments, a first light source is disposed apart from a second light source on the surface of the head-mounted display. In some embodiments, the plurality of light sources comprises locations on a map of the visual field.

In some embodiments, the head-mounted display comprises an eye tracker configured to measure a point of gaze of the subject. In some embodiments, the eye tracker comprises an optical tracking system. In some embodiments, the optical tracking system is adapted to record a corneal reflection. In some embodiments, the optical tracking system is adapted to record a pupil position. In some embodiments, the head-mounted display comprises a head-motion tracking system. In some embodiments, the head-mounted display comprises one or a plurality of light sources configured to control ambient light level.

In some embodiments, a response to the stimulus is characterized by a change from a first point of gaze to a second point of gaze. In some embodiments, a response to the stimulus is characterized by a press of a button. In some embodiments, a response to the stimulus is characterized by electroencephalography.

In some embodiments, the apparatus is configured to characterize a state or condition of the eye of the subject. In some embodiments, the state or condition of the eye of the subject comprises glaucoma. In some embodiments, the state or condition of the eye of the subject comprises stroke. In some embodiments, the state or condition of a subject eye comprises brain tumor. In some embodiments, the state or condition of a subject eye comprises a rate of vision loss. In some embodiments, the state or condition of each eye is characterized separately. In some embodiments, the state or condition of both eyes is characterized simultaneously.

In some embodiments, the apparatus further comprises an ophthalmoscope. In some embodiments, the apparatus is portable. In some embodiments, the apparatus comprises one or a plurality of lens, the one or a plurality of lenses configured to focus light onto a retina of the subject. In some embodiments, the apparatus does not include a lens. In some embodiments, a set of visual field data is configured to be transmitted to a mobile device. In some embodiments, the mobile device is configured to provide a map of the visual field. In some embodiments, the map of the visual field is configured to be provided to the subject or a third party.

In another aspect, a system is disclosed. The system may comprise the apparatus of any embodiment disclosed herein and a mobile device. In some embodiments, the mobile device is a tablet.

In another aspect, an apparatus for measuring a visual field of a subject is disclosed. In some embodiments, the apparatus may comprise an enclosure, wherein the enclosure surrounds at least one eye of the subject; a display comprising a distance from the eye of the subject to a surface of the display, wherein the distance is substantially constant along a portion of the surface of the display; and a plurality of light sources disposed on the display, wherein the light sources comprise a plurality of stimuli for measuring the visual field of the subject, wherein the apparatus may be worn by the subject

In some embodiments, the surface of the display may comprise a dome shape. In some embodiments, the surface of the display may comprise a shape which is hemispherical. In some embodiments, the surface of the display may comprise a shape which is a portion of the surface of a sphere. In some embodiments, the apparatus may be monocular. In some embodiments, the apparatus may be binocular. In some embodiments, the apparatus may be light sealed. In some embodiments, the apparatus may be semitransparent. In any of the preceding embodiments, the display may comprise a plurality of displays.

In some embodiments, the plurality of light sources may comprise light emitting diodes. In some embodiments, the display may not comprise a back-light. In some embodiments, the plurality of light sources may be stationary relative to the surface of the display. In some embodiments, the plurality of light sources may be mobile relative to the surface of the display.

In some embodiments, the plurality of light sources may additionally comprise a plurality of fixation points. In some embodiments, illumination of a first light source may comprise a first fixation point, illumination of a second light source may comprise a stimulus, and the second light source may comprise a second fixation point after the first light source is extinguished. In some embodiments, a first light source may be disposed apart from a second light source on the surface of the display. In some embodiments, the plurality of light sources may comprise locations on a map of the visual field.

In some embodiments, the head-mounted display may comprise an eye tracker configured to measure a subject point of gaze. In some embodiments, the eye tracker may comprise an optical tracking system. In some embodiments, the optical tracking system may be adapted to record a corneal reflection. In some embodiments, the optical tracking system may be adapted to record a pupil position. In some embodiments, the head-mounted display may comprise a head-motion tracking system. In some embodiments, the head-mounted display may comprise one or a plurality of light sources configured to control ambient light level.

In some embodiments, a response to the stimulus may be characterized by a change from a first point of gaze to a second point of gaze. In some embodiments, a response to the stimulus may be characterized by a press of a button. In some embodiments, a response to the stimulus may be characterized by electroencephalography.

In some embodiments, the apparatus may be used to characterize a state or condition of a subject eye. The apparatus may be used to characterize a classification of normal or abnormal visual function. In some embodiments, the state or condition of a subject eye may comprise a diagnosis of glaucoma. In some embodiments, the state or condition of a subject eye may comprise a diagnosis of stroke. In some embodiments, the state or condition of a subject eye may comprise a diagnosis of brain tumor. In some embodiments, the state or condition of a subject eye may comprise a rate of vision loss. In some embodiments, each eye may be characterized separately. In some embodiments, both eyes may be characterized simultaneously.

In some embodiments, the apparatus may comprise an ophthalmoscope. In some embodiments, the apparatus may be portable. In some embodiments, the apparatus may comprise one or a plurality of lens, which may focus light onto a subject retina. In some embodiments, the apparatus may not include a lens. In some embodiments, a set of visual field data may be transmitted to a mobile device. In some embodiments, the mobile device may be configured to provide a map of the visual field. In some embodiments, the map of a visual field may be provided to a user.

In another aspect, a method of measuring a visual field of a subject is provided. In some embodiments, the method comprises providing a head-mounted display comprising a plurality of light sources thereon, wherein the head-mounted display encloses a subject eye; wherein the head-mounted display comprises a distance from a plurality of locations on the display to the subject eye, the distance comprising a radius of an arc along a surface of the display; illuminating a first light source, wherein the first light source comprises a first fixation point; illuminating a second light source, wherein the second light source comprises a first stimulus; recording a response to a first stimulus; and illuminating a third light source.

In some embodiments, the third light source may comprise the second light source, and the second light source may comprise a second fixation point after the first light source may be extinguished. In some embodiments, the method may comprise extinguishing the first light source. In some embodiments, the method may comprise extinguishing the second light source. In some embodiments, the third light source may be displaced away from the first light source and the second light source, and the third light source may comprise a second fixation point after the first light source is extinguished.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of an apparatus for measuring a visual field, in accordance with some embodiments.

FIG. 2 shows a schematic of a display of an apparatus for measuring a visual field, in accordance with some embodiments.

FIG. 3 shows an example view of a display with multiple light sources as seen by a user, in accordance with some embodiments.

FIG. 4 shows an example of a method of testing a subject visual field, in accordance with some embodiments.

FIG. 5 shows an example of a headset for measuring a visual field, in accordance with some embodiments.

FIG. 6 shows an example of a digital processing device which may be programmed or otherwise configured to control an apparatus for testing a subject visual field.

### DETAILED DESCRIPTION

Many optic nerve diseases are left undiagnosed. For example, glaucoma, which affects approximately 3 million people in the United States, may be left undiagnosed in about 50% of its cases. When untreated, glaucoma may cause irreversible vision loss in its patients at a rate that is 10 times faster than among healthy individuals. Because glaucoma may initially affect peripheral vision, an example method for monitoring the progress of glaucoma is to monitor the visual field of a subject. Apparatuses and methods disclosed herein may improve upon existing methods for monitoring the progress of glaucoma by increasing the ease of performing and availability of visual field testing.

In some instances, a visual field of a subject may be measured by having the subject look at a point (also referred to as a “fixation point”) and displaying a first stimulus in the peripheral vision of the subject. The subject may then indicate if they have seen the first stimulus (e.g. by pressing a button or other user input). Alternatively or in addition, a change in direction of a gaze may be measured. Subsequently, a new, second stimulus may be displayed in a different location of the peripheral vision of the subject. The first stimulus may now become a fixation point, and the subject may indicate if they have seen the second stimulus by changing gaze and/or by use of a non-ocular indicator, such as a button.

Positive and/or negative responses to the stimuli may be recorded to map a visual field of the subject. Stimuli may be presented sequentially in different locations of the peripheral vision of the subject until the visual field of the subject has been sufficiently mapped. Since peripheral vision may be affected by contrast, the brightness of a stimulus relative to the ambient light level may be varied. The raw map of the visual field may be normalized (e.g., to generate a normalized map) and compared to baseline values for a healthy eye and/or compared to other measurements of the subject's visual field over time. Using this map of normalized values and the progression of visual field loss, and optionally in conjunction with other tests, it may be possible to diagnose, monitor, and treat diseases of the visual system and the optic nerve, such as glaucoma, stroke, brain tumor, vision loss, and other diseases.

In some instances, visual field testing may be improved by incorporating an eye tracking system, thereby reducing the amount of supervision required from a skilled technician to perform the test reliably. For example, the eye tracking system may replace (or be used in conjunction with) the skilled technician. In visual field testing where a subject manually indicates if they have seen a stimulus, the presence of a trained technician may be beneficial to improve testing results. For example, in a test where the subject focuses on a fixed point, a trained technician may monitor the subject for loss of focus, provide feedback, and adjust testing parameters. In another example, the technician may observe that the subject clicks too often or is rotating their head, in which case the technician may provide feedback and/or adjust testing parameters like the patient's position. An automated eye tracking system may not require a subject to manually indicate a response or a technician to adjust testing parameters based on observing the subject, thus removing the need for a skilled technician.

The present disclosure provides improved apparatuses, methods, and systems for measuring a visual field of a subject by providing a display system for measuring the visual field of the subject. A visual field test may be performed by sequentially providing optical stimuli (e.g. lights, other illuminations, etc.) in a plurality of different locations and/or different intensities while tracking the subject's gaze. A measured change in a subject gaze, following a stimulus, may be used to determine whether or not the subject has seen the stimulus. Based upon what the subject can and cannot see, the device may create a map of the visual field. Systems and methods of the present disclosure may use a head-mounted display to simplify, and/or improve accuracy (and precision) of, the visual field test.

### System for Measuring a Visual Field

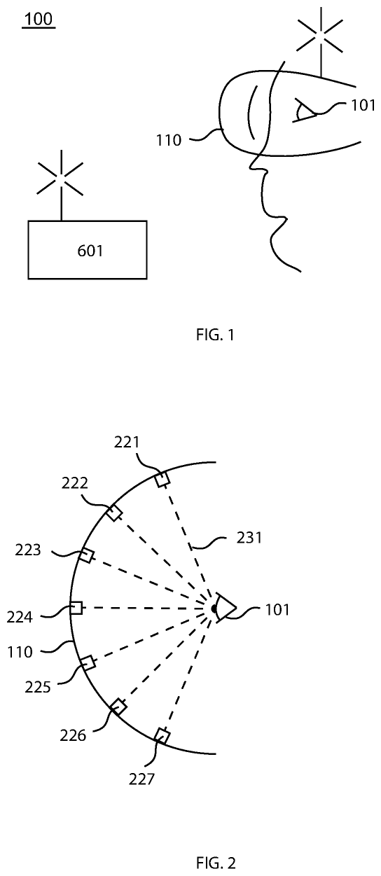

FIG. 1 shows an example of an apparatus for measuring a visual field. In an aspect, an apparatus for measuring a visual field is provided. The apparatus may comprise a head-mounted display 100. The apparatus may comprise a display portion 110 and an eye tracker, which may be incorporated into a head-mounted display 100. The head-mounted display may be a head-worn encapsulation or headset. The apparatus may comprise a head-mounted display wearable by a user. The head-mounted display may take the form of a goggle, a helmet, or any other form or shape. The head-mounted display may be sufficiently lightweight so as to be comfortably worn by a user. The head-mounted device may be portable.

The head-mounted display may be communicatively connected to a digital processing device or processor. For example, the digital processing device may be integrated in the head-mounted display. Alternatively, the digital processing device may be independent and/or located remotely from the head-mounted display. The head-mounted display and the digital processing device may be coupled via wired or wireless methods. For example, the digital processing device may be wirelessly connected to the head-mounted display via one or more wireless networks (e.g., via Bluetooth, via Internet, via Near Field Communication (NFC), etc.).

FIG. 5 shows an exploded view of an example head-mounted display 100 comprising display portions 110A and 110B. It will be understood that a head-mounted display may be used in its assembled state (i.e. with cap 510 flush with baffle 550 and elements 520, 530, 540A and 540B internal to the head-mounted display 100). It will be understood that circular elements for each eye may be concentric when assembled (i.e. display 110A may be concentric with lens 540A in front of the left eye and display 110B may be concentric with lens 540B in front of the right eye). The head-mounted display 100 may comprise a display portion 110. In an example, the display portion comprises a hemi-spherical or dome shape. In an additional example, the display portion comprises a spherical cap. A spherical cap may comprise a surface defined by a portion of the surface of a sphere. A spherical cap may be defined by an angle in steradians subtended by the display portion. A patient eye may be located at the center of the hemisphere or dome or at the apex of the solid angle subtended by the spherical cap.

In some instances, the display portion 110 of the head-mounted display 100 may be configured such that a first portion of the display provides a substantially constant distance between different locations on the display portion and the subject eye. For example, the hemi-spherical or dome shaped display portion may be positioned such that a subject eye is located substantially proximate to a center of the hemisphere or dome shape. In some cases, the lens may be cylindrically symmetric and comprise a single axis wherein the eye is approximately equidistant from the display. In cases where the display portion comprises a spherical cap, the radius of the sphere may comprise a distance from the patient eye to the display portion. The distance from the eye to the display may be from 5 to 9 centimeter (cm). Additionally or alternatively, the distance from the eye to the display may comprise a length of less than about 1 cm, 2 cm, 3 cm, 4 cm, 5 cm, 6 cm, 7 cm, 8 cm, 9 cm, 10 cm, 12 cm, 15 cm, 20 cm, 25 com, 30 cm, 50 cm, 100 cm, or more.

The solid angle subtended by the spherical cap may be from 0 steradians to 4π steradians. In an example, the solid angle subtended by the spherical cap may be less than about 0.01 steradians, 0.1 steradians, π/16 steradians, π/8 steradians, π/4 steradians, π/2 steradians, π steradians, 3π/2 steradians, 2π steradians (hemispherical), 5π/2 steradians, or 4π steradians.

In contrast to systems with flat screens where the distance between a subject eye 101 and different locations on a flat screen is variable, the display 110 may comprise a substantially constant distance between the subject eye and different locations on the display. The distance may be substantially constant over the surface defined by the display portion. For example, the distance may be substantially constant to within about 5 millimeters (mm). In another example, the distance between a subject eye and the display may be substantially constant to within at most about 500 microns, 1 mm, 2 mm, 3 mm, 4 mm, 5 mm, 6 mm, 7 mm, 8 mm, 9 mm, 1 centimeter (cm), 2 cm, 3 cm, 4 cm, 5 cm, or more. Alternatively or in addition, the distance may be substantially constant to within a range between any two of the preceding values.

Screens which are flat may compromise test variability in exchange for decreased hardware development time and/or cost and increased flexibility. The distance between the eye and different lights on the flat screen is variable, and the light source on the screen may not necessarily be directed toward the subject eye. Therefore, if a first light is shined at a specific intensity at the center of the screen and a second light is shined at another location of the screen at the same intensity, the intensity of the two lights that reaches the retina of the subject's eye may be different. This may increase difficulty in comparing vision loss in one area of a subject's eye to another area of the subject's eye. An accurate map of vision loss at various areas of a subject's eye may be important to accurately diagnose glaucoma. Mathematical corrections for variations in distance may introduce additional sources of error. Providing a display system with substantially constant distance to the eye can address these problems.

The display portion 110 may be monocular; alternatively, the display portion may be binocular. The display portion 110 may have any number of portions of the display, each portion configured to provide a substantially constant distance between different location on each portion and the subject eye, such as 1 (e.g., entire display provides substantially constant distance), 2, 3, 4, 5, 6, 7, or more portions. In some instances, the display portion 110 may be configured such that a first portion of the display provides a substantially constant distance between different locations on the portion and a first subject eye (e.g., left eye) 110A, and a second portion of the display portion of the display provides a substantially constant distance between different locations on the another portion and a second subject eye (e.g., right eye) 110B. The display portions may be disposed in a holder 520, which may be configured to integrate into the rest of the head-mounted display.

The head-mounted display 100 may be baffled to decrease external light leaks toward the eye 101. The head-mounted display may comprise an enclosure to surround a subject eye. The display portion may be light-sealed. The display portion may be transparent or semitransparent. The head-mounted display may comprise a baffle 550. The baffle 550 may be padded to provide comfort to a subject over an extended period of wear. The baffle may be transparent or semitransparent. The baffle may be opaque. The baffle may be light-sealed. The baffle may be connected to a restraint system which may secure the baffle to a patient head. The restraint system may comprise for example a strap. The restraint system may be adjustable to fit a wide range of subject head sizes. The head-mounted display may comprise a cap 510. The cap may secure parts of the display together. The cap may be transparent or semi-semitransparent. The cap may be opaque. The cap may be light-sealed. The baffle and the cap together may sufficiently seal a subject eye, such that a test of a subject visual field may conducted in near-darkness.

Head-mounted display 100 may additionally comprise an eye-holder 530. The eye holder 530 may aid in securing and centering a display portion on a patient eye. For example, the eye holder may be adjusted such that the length between centers of sockets in the eye-holder may comprise the distance between the subject's eyes. The eye holder, when adjusted, may move the center of the right and left display portions and right and left lens elements such that they are substantially concentric with a patient eye. The intraocular distance, as set by the eye holder may be adjusted by a subject and/or another user. This adjustment may conducted using an interface external to the display. The interface may comprise a knob, dial, switch, button, or other interface.

Head-mounted display 100 may additionally comprise one or a plurality of lens elements 540. In some embodiments, a system for measuring a subject visual field may not comprise a lens element. The lens elements 540 may have any number of lens or compound lenses. Each lens may configured to focus light on a patient retina. In some instances, the lens elements 540 may be configured such that a first lens focuses light to a first subject eye (e.g., left eye) 540A, and a second lens element may be configured such that a first lens focuses light to a first subject eye (e.g., right eye) 540B. The lens elements may be disposed in a holder, which may be configured to integrate into the rest of the head-mounted display. The focal length of the one or a plurality of lens may comprise a distance between the display portion and patient retina. The focal length of each element may be individually fine-tuned by a subject or another user to optimize the focus. This tuning may conducted using an interface external to the display. The interface may comprise a knob, dial, switch, button, or other interface. The interface for adjusting the focus of each lens may be integrated with the interface for adjusting an intraocular distance.

A lens may be advantageous to focus light on a retina if the subject eye is not effective at focusing on its own, which may result in blurred vision. With minimal background lighting and lower intensity light stimuli, blurring may be reduced relative to other systems and methods. Additional cost savings may be provided by a reduced need for multiple lens elements with varying focusing conditions.

It will be understood that the head-mounted display 100 may comprise additional elements, described elsewhere herein. Such elements include electronic elements, such as a wireless radio, a mobile processing unit, light sources, eye-tracking hardware, etc.

FIG. 2 shows a schematic of a display 110 of an apparatus 100 for measuring a visual field. The apparatus may comprise a plurality of light sources 221, 222, 223, 224, 225, 226, and 227. In the illustrated embodiment, the light sources may be located at a distance 231 from the eye 101 of a subject, which may be substantially constant across one axis of the display. In such an embodiment, a plurality of light sources may be pointed towards the subject's eye and at a similar distance from it. This may enable a visual field test using the apparatus 100 to directly compare visual field defects and vision loss in a plurality of areas of the eye. In some embodiments, the plurality of areas may be measured in a single test.

A visual field test may additionally generate a map of a subject vision as a function of a relation between the brightness of the light source and that of the background. A relation may comprise a ratio or a differential between the two values. A relation may comprise a measurement of the contrast. An example system that measures contrast may comprise a backlight. For example, in a system without an enclosure, the backlight may be varied to control the luminance contrast in various ambient lighting conditions. Efforts to vary the backlight may mitigate variabilities in ambient lighting conditions, but these variabilities may not be entirely compensated.

In another example, the display may not comprise a backlight. A backlight may not be necessary when the system for measuring a visual field encloses a subject eye. In this example, the interior of the headset may not be significantly affected by ambient lighting conditions. The contrast of the stimuli may be determined by measuring the inner headset light intensity (e.g. with a photodiode) and comparing it to the intensity of the light stimuli being displayed. This may simplify the display system and, additionally or alternatively, may decrease cost by removing the backlight. Additionally, the above example may decrease cost by removing need for a lens to focus a light source on a subject eye.

In another example, the display may comprise methods to vary the apparent size of the light sources. Varying the size of the light sources may contribute to a decrease in blur or haze around the light sources.

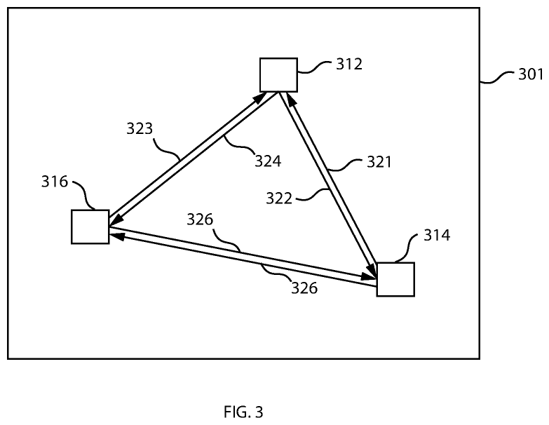

FIG. 3 shows a view of a display with multiple light sources as seen by a user. A system for measuring a visual field of a subject may reduce the number of components and minimize space taken by the headset. This may be accomplished by mapping the visual field based on a relative position of the various light sources. To map a visual field, the exam may test whether the subject can see a second light source when looking at a first light source. The relative position between the two light sources may be used to determine what points of the visual field can and cannot be seen by a subject. In an example, assume there are three light sources. If the subject were looking at light source 312, light source 314 or light source 316 may be turned on. At which point the subject may change gaze position from light source 312 to light source 314 or light source 316. A similar effect may occur if the subject were initially looking at light source 314 or light source 316. The relative distance between each light source will be different and correspond to a different point in a subject's visual field. This may allow for 3 light sources to test 6 points in the visual field, as indicated by the arrows 321, 322, 323, 324, 325, and 326 in FIG. 3. Using a typical method with a static focal point, the three light sources may only be able to test two points. Using relative position, a significantly larger number of points may be tested with fewer light sources.

In some embodiments, the system for testing a visual field comprises 5 to 40 light sources. Alternatively, the system for testing a visual field may comprise more than about 2, 3, 4, 5, 10, 20, 30, 40, 50, 54, 100, 200, 500, 1000, 2000, 5000, 10000, 20000, 50000, 100000, 200000, 500000 light sources, or any number of light sources defined by a range between any two of the preceding values. The visual field test may use less than the total number of light sources.

The light sources may have the same or different characteristics such as polarization, intensity, power, luminous intensity, wavelength, range of wavelengths (e.g., bandwidth), repetition rate, pulse duration, and mode. A light source may be configured to direct light with an intensity of less than about 1,000,000 millicandelas (mcd), 100,000 mcd, 10,000 mcd, 1,000 mcd, 10 mcd, 1 mcd, 1 microcandela (ucd), or even less. The light sources may comprise an intensity which may be variable. The light sources may comprise an intensity which is fixed. The light sources may be white light sources. The white light sources may comprise a broad or a narrow spectral band. Alternatively, the light sources may comprise a color within a human visual range. The light sources may comprise light sources of all the same color or may comprise subsets of light sources with a plurality colors. In some embodiment, the color of an individual light source may vary. The light sources may be about 400 nanometers (nm), 450 nm, 500 nm, 550 nm, 600 nm, 650 nm, 700 nm, 750 nm, 800 nm, or any wavelength within a range defined by any two the preceding values. Additionally or alternatively, the light sources may comprise a bandwidth of about 1 nm, 5 nm, 10 nm, 50 nm, 100 nm, 400 nm, or any bandwidth within a range defined by any two of the preceding values.

Alternatively, a visual field test may use all of the light sources. In some embodiments, the light sources may comprise light emitting diodes (LEDs). In some embodiments, the light sources are mobile relative to a surface of the display. In some embodiments, the light sources may be removably placed in varying positions relative to the display. In some embodiments, one or a plurality of light sources may be used to control the ambient light level. In some embodiments, one or a plurality of light sources may be used to provide a reference illumination system.

In some embodiments, a system for measuring a visual field of a subject may comprise an eye tracker. In some embodiments, the eye tracker may track an eye movement by an optical system. In some embodiments, the optical system may record a pupil position, reaction time, cornea size, and size. Additionally or alternatively, the optical system may record changes in a corneal reflection. The eye tracker may comprise a commercial eye tracking system from Pupil Labs®, SMI®, Fove®, etc. In some embodiments, one or a plurality of light sources may generate light to reflect off of a subject cornea. The apparatus for measuring a visual field may comprise one or a plurality of cameras or sensors internal to the headset for tracking a subject gaze. Other methods of tracking the movement of a subject eye may be possible. The eye tracking system may additionally comprise a head-motion tracking system, such as, for example, by incorporating an accelerometer. The head-motion tracking system may comprise a camera system exterior to the head-mounted display which may record motion of fiducial markers on the head-mounted display or on the subject head. In some embodiments, the system for measuring a visual field does not comprise an eye tracking system. In some embodiments, a response to a stimulus is recorded by the press of a button, switch, or other mechanical indication. In some embodiments, a response to a stimulus is registered by electroencephalography.

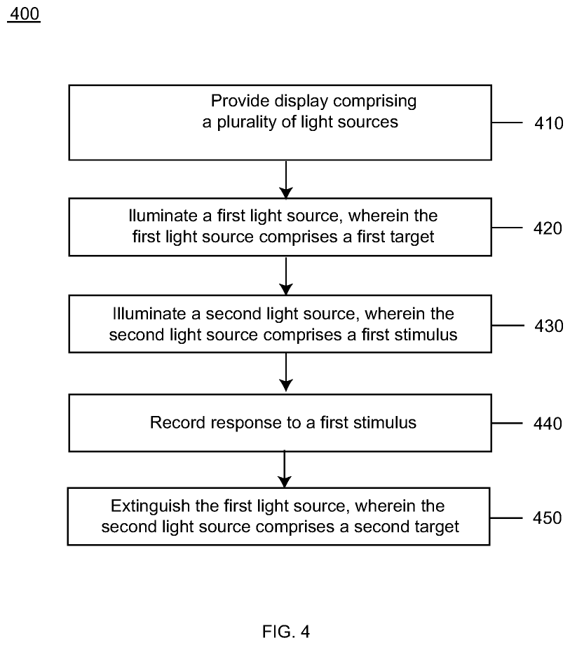

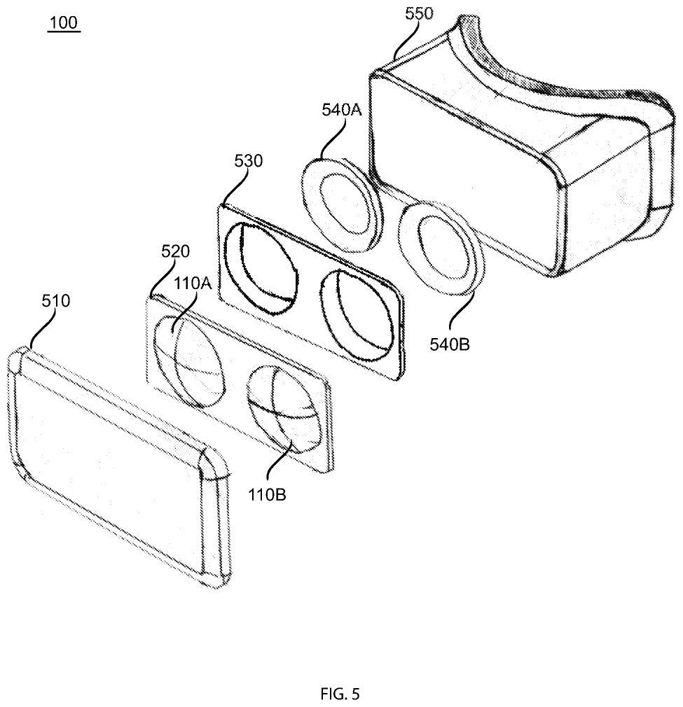

FIG. 4 shows an example of a method 400 of testing a subject visual field. At an operation 410 a method 400 may comprise providing a head-mounted display comprising a plurality of light sources. A method 400 may comprise selection of a type of test to perform. Selection of a test to perform may comprise selection on the headset itself or using a connected device such as phone, tablet, local computer, or remote computer. In some embodiments, selection of a test to perform may comprise use of a digital processing device as disclosed herein, which device may not be connected. A method may comprise placing a headset on a subject head. At an operation 420, the method 400 may comprise illumination of a first light source, which may comprise a first fixation point. A subject gaze may be measured. If the subject gaze is at the first light source, a timer may be started. At an operation 430, the method 400 may comprise illuminating a second light source, which may comprise a first stimulus. At an operation 440, the method 400 may comprise recording a response to the first stimulus. If the subject gaze changes direction from the first light source to the second light source, a timer may be stopped and a data point recorded. If no change of direction is recorded within a set time, a data point may be recorded specifying no gaze change has occurred. For example, if the subject looks at the second light, then the processor may store the fact that the user saw the light. The processor may store how much time it took the subject to respond to the stimulus. For example, if the subject does not see the light, data about the light being not seen may be stored. In some instances, the data may be binary (e.g., 0/1, T/F, etc.). In other instances, the data may not be binary; for example, a response time may be recorded for instances in which the light is seen. At an operation 450, the method 400 may comprise extinguishing the first light source and/or the second light source. If the second light source is left on, the second light source may comprise a second fixation point and a second stimulus may be displayed by illuminated a third light source. In some cases at an operation 450, the third light source may comprise a second fixation point, and a fourth light source may be illuminated which may comprise the second stimulus. Operations 430 to 450 may be repeated with a third light source. The subject's visual field may be tested with different lights of different intensities and locations. A plurality of type of results may be displayed such as for example as a representation of the visual field of the subject.

The visual field may comprise a number of illumination steps. The number of illumination steps may be 2, 5, 10, 20, 50, 100, 200, 500, 1000, 2000, 5000, 10000, or any number of illumination steps within a range defined by any two of the preceding values. In some embodiments, the visual field test comprises a single fixation point. In some embodiments, the visual field test comprises a fixation point that changes from light source to light source.

Optionally, a visual field test may comprise varying the intensity of one or more light sources. Optionally, a visual field test may comprise an initial calibration step. The test may comprise varying the order of illumination of the various light sources. The test may comprise moving light sources. The visual field test may comprise strobing or variation in the intensity of a light source. The visual field test may comprise changing the color of one or more light sources. The visual field test may comprise varying backlight intensity. The visual field test may comprise varying the duration in which a light source is illuminated. The visual field test may comprise focusing light into a subject eye. The visual field test may comprise selecting a lens to improve focus onto a subject eye.

Visual field tests may be recorded longitudinally and/or periodically over a period of days, months, and/or years. For example, a visual field test may be measured once per week, once per month, once per quarter, once per year, or at a frequency within a range between any two of the preceding values. Alternatively, visual field tests may be recorded irregularly over any duration of time. Changes in a subject visual field may be measured over time, for example, in order to track the progress of disease. Various types of data representations may be displayed such as a representation of a visual field, rate of vision loss, etc.

Data may be displayed to a user and/or to a skilled technician. Information may be made available immediately subsequent to the test or after a short period of time. Data may be stored locally on a connected device or on a remote processor. Stored data may be presented to a user and/or a skilled technician at any time. Determining test result validity may be carried out by a skilled technician or with the aid of a classifier model. Alternatively, data may be analyzed entirely by a classifier model. The classifier model may be implemented by a processor such as on board the connected device, on a local processor, or by a processor disclosed herein. Additionally, the test results can be sent to physicians who can perform a classification.

The classification may be made by a physician who is remote to the system for measuring a visual field. For example, the data may be provided to the physician on the processor, which may be a connected device or a remote device as described elsewhere herein.

The apparatus may be used to characterize a state or condition of the eye. For example, a state or condition of the eye may be characterized by a narrowing of the visual field. In another example, a state or condition of the eye may be characterized by a loss of sensitivity to changes in contrast, speed of response, changes in size of stimulus, etc. The state or condition of the eye may be a function of a location within the visual field. Optionally, the analyzed data may be used to diagnose ocular or neurological disorder. A state or condition of the eye may comprise a classification of glaucoma. A state or condition of the eye may comprise a classification of stroke. A state or condition of the eye may comprise a classification of brain tumor. A state or condition of the eye may comprise a classification of vision loss. Characterizing a state or condition of the eye may comprise characterizing each eye separately. Characterizing a state or condition of the eye may comprise characterizing both eyes simultaneously. A state or condition of the eye may comprise a classification of normal or abnormal visual function.

### Digital Processing Device

In some embodiments, the systems, apparatuses, and methods described herein include a digital processing device, or use of the same. The apparatus for testing a visual field may be connected to a digital processing device by a wired connection or a wireless connection. The digital processing device may be connected to the apparatus for testing a visual field via the Internet, via Bluetooth, via short range infrared, via a cellular connection, etc.

In some embodiments, the digital processing device may comprise a non-transitory computer readable storage medium with instructions stored thereon. The instructions may be implemented by a processor disclosed herein. The instructions when executed may configure the headset to implement the methods disclosed herein. Operations of the methods disclosed herein may be controlled by a digital processing device. The digital processing device may be remote to a user. The digital processing device may be the mobile device of a user. The apparatus may be connected to one or more digital processing devices such as the device of a subject and the device of another user. The other user may be a medical provider.

In some embodiments, each of the one or more digital processing devices may perform only a portion of the methods disclosed herein. For example, a subject digital processing device may be used to control test operations while another digital processing device, such as a third party (e.g. medical professional) digital processing device may be used to analyze the visual field data. The third party (e.g. medical professional) may then provide a classification to a subject.

In further embodiments, the digital processing device includes one or more hardware central processing units (CPUs), general purpose graphics processing units (GPGPUs), or field programmable gate arrays (FPGAs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, and media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user (e.g. subject), technician (e.g. clinician), or third-party user. In some embodiments, the display included in the digital processing device is a system for testing a subject visual field of the present disclosure; however, the display included in the digital processing device may be a display configured to send visual information to a technician or observer. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user (e.g. subject), technician (e.g. clinician), or third-party user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect®, Leap Motion®, or the like. In further embodiments, the input device is a headset, such as a virtual reality headset. In some embodiments, the headset is an Oculus Rift®, Samsung Gear VR®, HTC Vibe®, Sony Playstation VR®, or Google Daydream®. In some embodiments, the headset is an augmented reality headset, such as Google Glass®. In still further embodiments, the input device is a combination of devices such as those disclosed herein.

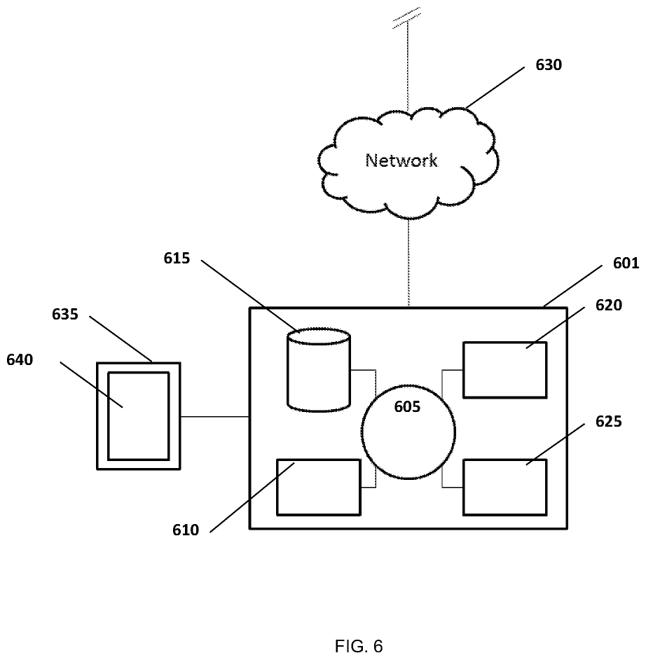

Referring to FIG. 6, in a particular embodiment, an example of a digital processing device 601 is programmed or otherwise configured to control a system for testing a subject visual field as described herein. The device 601 can regulate various aspects of the system for testing a visual field of the present disclosure, such as, for example, performing processing steps. In this embodiment, the digital processing device 601 includes a central processing unit (CPU, also “processor” and “computer processor” herein) 605, which can be a single core or multi core processor, or a plurality of processors for parallel processing. The digital processing device 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The digital processing device 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the device 601, can implement a peer-to-peer network, which may enable devices coupled to the device 601 to behave as a client or a server.

Continuing to refer to FIG. 6, the CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and write back. The CPU 605 can be part of a circuit, such as an integrated circuit. One or more other components of the device 601 can be included in the circuit. In some cases, the circuit is an application specific integrated circuit (ASIC) or a field programmable gate array (FPGA).

Continuing to refer to FIG. 6, the storage unit 615 can store files, such as drivers, libraries and saved programs. The storage unit 615 can store user or subject data, e.g., user or clinician preferences, user programs. The digital processing device 601 in some cases can include one or more additional data storage units that are external, such as located on a remote server that is in communication through an intranet or the Internet.

Continuing to refer to FIG. 6, the digital processing device 601 can communicate with one or more remote computer systems through the network 630. For instance, the device 601 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610. The digital processing device may comprise a display 640, which may be configured to display information to a user via user-interface 635 displayed thereon.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein. In some embodiments, the mobile device may be configured to control the system for testing a visual field as described herein. In some embodiments, the mobile device may be configured to process data from a subject visual field test or allow data from a subject visual field test to be viewed by a remote user, subject, or technician.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, and Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone, and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. In some embodiments, a computer program may control the operation of a system for testing a subject visual field. In some embodiments, a computer program may analyze subject data from a test of a visual field. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable compiled applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An apparatus for measuring visual function across a visual field of a subject comprising:
an enclosure, wherein the enclosure is configured to surround at least one eye of the subject;
a display comprising a surface, wherein the surface is configured to be spaced a distance from the eye of the subject, wherein the distance is substantially constant along a portion of the surface of the display; and
a plurality of light sources disposed on the display, wherein the light sources comprise a plurality of stimuli for measuring the visual field of the subject,
wherein the apparatus is configured to be worn by the subject.

2. The apparatus of claim 1, wherein the surface of the display comprises a dome shape, a shape which is hemispherical, or a shape which is a portion of a surface of the sphere.

3. (canceled)

4. (canceled)

5. The apparatus of claim 1, wherein the apparatus is monocular or binocular.

6. (canceled)

7. The apparatus of claim 1, wherein the apparatus is light sealed.

8. The apparatus of claim 1, wherein the apparatus is semitransparent.

9. The apparatus of claim 1, wherein the display comprises a plurality of displays.

10. (canceled)

11. The apparatus of claim 1, wherein the display does not comprise a back-light.

12. The apparatus of claim 1, wherein the plurality of light sources is stationary relative to the surface of the display.

13. The apparatus of claim 1, wherein the plurality of light sources is mobile relative to the surface of the display.

14. The apparatus of claim 1, wherein the plurality of light sources additionally comprises a plurality of fixation points and wherein the plurality of light sources comprises locations on a map of the visual field.

15. The apparatus of claim 14, wherein illumination of a first light source comprises a first fixation point, wherein illumination of a second light source comprises a stimulus, and wherein the second light source comprises a second fixation point after the first light source is extinguished, wherein the first light source is disposed apart from the second light source on the surface of the display.

16. (canceled)

17. (canceled)

18. The apparatus of claim 1, wherein the display comprises an eye tracker configured to measure a point of gaze of the subject.

19. The apparatus of claim 18, wherein the eye tracker comprises an optical tracking system and wherein the optical tracking system is adapted to record a corneal reflection or a pupil position.

20. (canceled)

21. (canceled)

22. The apparatus of claim 1, wherein the display comprises a head-motion tracking system.

23. The apparatus of claim 1, wherein the display comprises one or a plurality of light sources configured to control ambient light level.

24. The apparatus of claim 15, wherein a response to the stimulus is characterized by a change from a first point of gaze to a second point of gaze.

25. The apparatus of claim 15, wherein a response to the stimulus is characterized by a press of a button.

26. The apparatus of claim 15, where a response to the stimulus is characterized by electroencephalography.

27. The apparatus of claim 1, wherein the apparatus is configured to characterize a state or condition of the eye of the subject and wherein the state or condition of the eye of the subject comprises glaucoma, stroke, brain tumor, or a rate of vision loss.

28. (canceled)

29. (canceled)

30. (canceled)

31. (canceled)

32. The apparatus of claim 27, wherein the state or condition of each eye is characterized separately or simultaneously.

33. (canceled)

34. The apparatus of claim 1, further comprising an ophthalmoscope.

35. (canceled)

36. The apparatus of claim 1, wherein the apparatus comprises one or a plurality of lenses, the one or a plurality of lenses configured to focus light onto a retina of the subject.

37. (canceled)

38. The apparatus of claim 1, wherein a set of visual field data is configured to be transmitted to a mobile device.

39. The apparatus of claim 38, wherein the mobile device is configured to provide a map of the visual field.

40. The apparatus of claim 39, wherein the map of the visual field is configured to be provided to the subject or a third party.

41. A method of measuring visual function across a visual field of a subject, the method comprising:
providing a head-mounted display comprising a plurality of light sources thereon, wherein the head-mounted display is configured to enclose an eye of the subject; wherein the head-mounted display is configured to be spaced a distance from the eye of the subject, the distance comprising a radius of an arc along a surface of the display;
illuminating a first light source, wherein the first light source comprises a first fixation point;
illuminating a second light source, wherein the second light source comprises a first stimulus;
recording a response to a first stimulus; and
illuminating a third light source.

42.-86. (canceled)
